(19) United States
(12) Patent Application Publication
(10) Pub. No.: US 2025/0320175 A1
(43) Pub. Date: Oct. 16, 2025

(54) Acetoacetate Based Ketals

(71) Applicant: ARXADA AG, Visp (CH)

(72) Inventors: Ulrich Mayerhoeffer, Visp (CH); Sebastian Benz, Visp (CH); Janis Michel, Visp (CH); Florian Dardano, Visp (CH); Marianne Haedener, Visp (CH); Stefan Ellinger, Visp (CH)

(21) Appl. No.: 19/040,931

(22) Filed: Jan. 30, 2025

Related U.S. Application Data
(62) Division of application No. 18/713,225, filed on May 24, 2024.

(30) Foreign Application Priority Data
Nov. 26, 2021 (EP) 21210901.1

(51) Int. Cl. C07C 69/72; C07C 67/42; C07C 67/46; C07D 319/06

(52) U.S. Cl. CPC C07C 69/72; C07C 67/42; C07C 67/46; C07D 319/06

(57) ABSTRACT

The present invention relates to polyol-derived compounds and processes preparing the same.

### TECHNICAL FIELD

The present invention relates to polyol-derived compounds and processes preparing the same.

### Technological Background

Acetoacetylated polyalcohols and p-hydroxy butyric acid (BHB) esters of polyalcohols prepared therefrom are valuable compounds with a versatile utilization for example as parenteral nutrients or for the treatment of certain diseases.

US 2019/117612 A1 pertains to the field of migraine headaches and the management of the symptomology thereof using 3-hydroxybutyrate glycerides.

US 2018/193300 A1 pertains to a method of treatment of mild to moderate non-penetrating closed traumatic brain injury and mild to moderate traumatic brain injury due to surgical intervention using 3-hydroxybutyate glycerides.

Acetoacetylated polyalcohols and p-hydroxy butyric acid (BHB) esters of polyalcohols are usually prepared by coupling a polyalcohol such as glycerol with protected p-hydroxy butyric acid or acetoacetate esters. Both methods suffer from poor atom economy and result in more waste.

Moreover, BHB esters of polyalcohols usually have a low BHB content per polyalcohol unit. However, in order to increase BHB delivery efficiency, a high BHB content per polyalcohol unit would be desirable. Furthermore, protecting the BHB units in BHB esters of polyalcohols would enable the delivery of further BHB precursors, which upon hydrolysis are oxidized by the body to BHB, which further increases BHB delivery efficiency.

Hence, there is a need for providing polyalcohols with a high BHB unit concentration per polyalcohol unit and in which the BHB units are protected.

There is further a need for optimized processes for the synthesis of such p-hydroxy butyric acid (BHB) esters of polyalcohols having a high content of BHB units and in which the BHB units are protected.

### SUMMARY OF THE INVENTION

The inventors surprisingly found that the processes according to the present invention by reacting a diketene with a polyol or a p-hydroxyl butyric acid ester of a polyol provides an excellent method for producing stable and neutral analogues of p-hydroxy butyric acid. The reaction of a polyol or a p-hydroxyl butyric acid ester of a polyol with diketene and subsequent ketal formation allows for facile access to the desired protected products. Moreover, the processes according to the present invention allow for the synthesis of polyalcohols with a high BHB unit concentration per polyalcohol unit.

Accordingly, the present invention provides a compound of formula 1

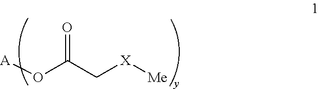

wherein
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁-12 alkyl, C₃-8 cycloalkyl, linear or branched C₁-12 hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A.

In another aspect, the present invention provides a compound of formula 9

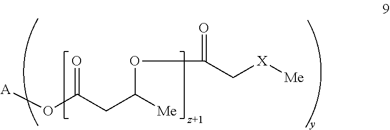

wherein
z is 0 or more,
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A.

In another aspect, the present invention provides a process for the preparation of a compound of formula

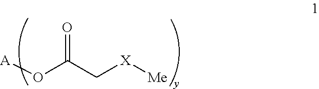

wherein
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A;
wherein the process comprises:
   (i) reacting an organic polyol of formula 2 with diketene 3 resulting in the formation of a compound according to formula 4;

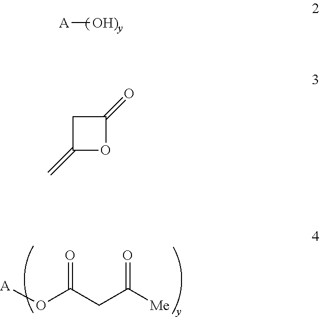

and
(ii) reacting the compound of formula 4 with an alcohol, a thiol, or a mercapto alcohol resulting in the formation of a compound according to formulas 5 to 8

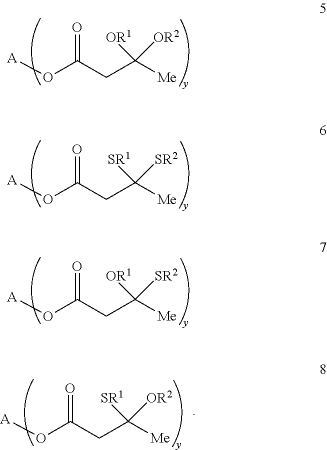

In another aspect, the present invention provides a process for the preparation of a compound of formula 9

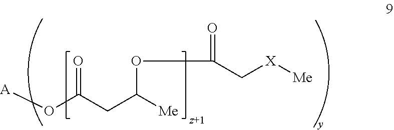

wherein
z is 0 or more,
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A;
wherein the process comprises:
   (i) reacting a compound of formula 10 with diketene 3 resulting in the formation of a compound according to formula 11;

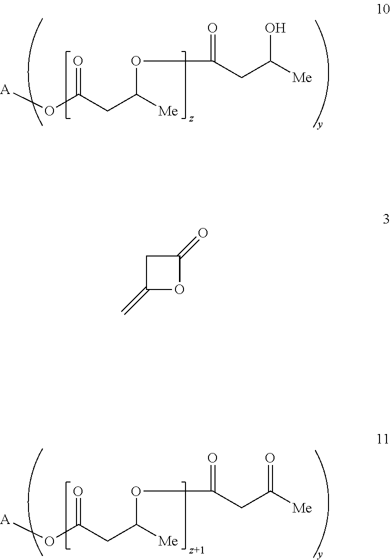

and
(ii) reacting the compound of formula 11 with an alcohol, a thiol, or a mercapto alcohol resulting in the formation of a compound according to formula 12 to 15

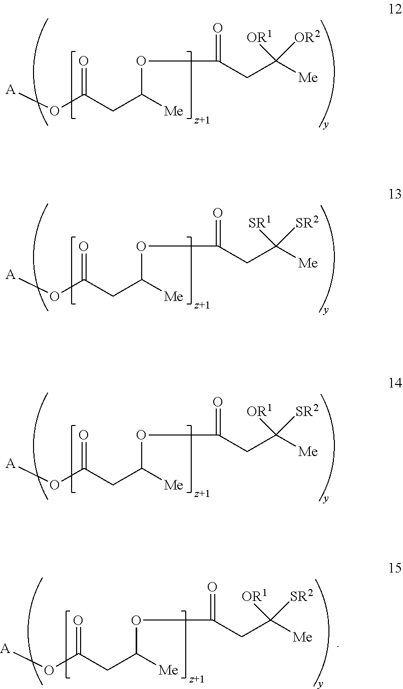

### DETAILED DESCRIPTION OF THE INVENTION

In the following, the invention will be explained in more detail.

### Definitions

In order for the present invention to be readily understood, several definitions of terms used in the course of the invention are set forth below.

According to the present invention, the term “linear or branched C₁₋₁₂ alkyl” refers to a straight-chained or branched saturated hydrocarbon group having 1 to 12 carbon atoms, such as 1, 2, 3, 4, 5, 6, 7, 8, 9, 10, 11 or 12 carbon atoms including methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethyl propyl, 1,2,2-trimethylpropyl, 1-ethyl-1-methylpropyl and 1-ethyl-2-methylpropyl.

According to the present invention, the term “C₃₋₈ cycloalkyl” refers to a monocyclic or polycyclic saturated hydrocarbon group having 3 to 8 carbon ring members including cyclopropyl, cyclobutyl, cyclopentyl, cyclohexyl, cycloheptyl, and cyclooctyl.

According to the present invention, the term “linear or branched C₁₋₁₂ hydroxyalkyl” refers to a straight-chained or branched saturated hydrocarbon group having 1 to 12 carbon atoms as defined above, wherein at least one hydrogen atom is replaced by a hydroxy group, including hydroxymethyl, 1-hydroxyethyl, 2-hydroxyethyl, 1-hydroxypropyl, 2-hydroxypropyl, 3-hydroxypropyl, 2-hydroxyisopropy, 1-hydroxybutyl, 2-hydroxybutyl, 3-hydroxybutyl, 4-hydroxybutyl, 1-hydroxypentyl, 2-hydroxypentyl, 3-hydroxypentyl, 4-hydroxypentyl, 5-hydroxypentyl, 1-hydroxyhexyl, 2-hydroxyhexyl, 3-hydroxyhexyl, 4-hydroxyhexyl, 5-hydroxyhexyl, 6-hydroxyhexyl, and 2-ethyl-1-hydroxyhexyl.

According to the present invention, the term “5 to 8 membered cyclic ketal” refers to monocyclic saturated acetals formed from the condensation of a diol with a ketone group. 5 to 8 membered includes 5-, 6-, 7-, and 8-membered rings. Suitable diols for the formation of 5 to 8 membered cyclic ketals include ethylenglycol, 1,2-propanediol, 1,2-dimethyl-1,2-propanediol, 1,3-propanediol, 2-methyl-1,3-propanediol, 2,2-dimethyl-1,3-propanediol, 1,3-butanediol, 2-methyl-1,3-butanediol, 2,2-dimethyl-1,3-butanediol, 1,2-dimethyl-1,3-butanediol, 2,3-butanediol, 2-methyl-2,3-butanediol, pinacol, 1,4-butanediol, and 1,5-pentanediol.

According to the present invention, the term “5 to 8 membered cyclic thioketal” refers to monocyclic saturated thioacetals formed from the condensation of a dithiol with a ketone group. 5 to 8 membered includes 5-, 6-, 7-, and 8-membered rings. Suitable dithiols for the formation of 5 to 8 membered cyclic thioketals include ethane-1,2-dithiol, 1,2-propanedithiol, 1,2-dimethyl-1,2-propanedithiol, 1,3-propanedithiol, 2-methyl-1,3-propanedithiol, 2,2-dimethyl-1,3-propanedithiol, 1,3-butanedithiol, 2-methyl-1,3-butanedithiol, 2,2-dimethyl-1,3-butanedithiol, 1,2-dimethyl-1,3-butanedithiol, 2,3-butanedithiol, 2-methyl-2,3-butanedithiol, 2,3-dimethyl-2,3-butanedithiol, 1,4-butanedithiol, and 1,5-pentanedithiol.

According to the present invention, the terms “5 to 8 membered 1,3-oxathiolane” refers to monocyclic saturated 1,3-oxathiolanes formed from the condensation of a mercapto alcohol with a ketone group. 5 to 8 membered includes 5-, 6-, 7-, and 8-membered rings. Suitable mercapto alcohols for the formation of 5 to 8 membered cyclic thioketals include mercaptoethanol, 3-mercapto-1-propanol, 1-mercaptopropane-2-ol, 2-mercaptopropane-1-ol, 3-mercapto-3-methylbutane-2-ol, 3-mercapto-2-methylbutane-2-ol, 1,3-propanedithiol, 3-mercapto-2-methylpropane-1-ol, 3-mercapto-2,2-dimethylpropane-1-ol, 4-mercaptobutane-2-ol, 1-mercaptobutane-3-ol, 3-mercaptobutane-1-ol, 3-mercapto-2-methylbutane-1-ol, 4-mercapto-3-methylbutane-2-ol, 4-mercapto-3,3-dimethylbutane-2-ol, 3-mercapto-2,2-dimethylbutane-1-ol, 4-mercapto-3-methylpentane-2-ol, 3-mercaptobutane-2-ol, 3-mercapto-2-methylbutane-2-ol, 3-mercapto-3-methylbutane-2-ol, 3-mercapto-2,3-dimethylbutane-2-ol, 4-mercaptobutane-1-ol, and 5-mercaptopentane-1-ol.

According to the present invention, the term “organic polyol” refers to a linear, branched, or cyclic organic compound with 2 to 18 carbon atoms having at least 2 hydroxyl groups. As such, the organic polyol may have 2, 3, 4, 5, 6, 7, 8, 9, 10, 11, 12, 13, 14, 15, 16, 17, or 18 carbon atoms. In one embodiment, the organic polyol may have 2, 3, 4, 5, 6, 7, 8, 9, 10, 11, 12, 13, 14, 15, 16, 17, or 18 hydroxyl groups. In one embodiment, no more than one hydroxyl group is connected to one carbon atom. In one embodiment, the organic polyol contains only carbon, hydrogen, and oxygen atoms.

It is to be understood that the linear or branched C₁₋₁₂ alkyl, C₃₋₈ cycloalkyl, linear or branched C₁₋₁₂ hydroxyalkyl, phenyl, 5 to 8 membered cyclic ketal, 5 to 8 membered cyclic thioketal, and the 5 to 8 membered 1,3-oxathiolane groups may optionally be further substituted. Exemplary substituents include hydroxy, linear or branched C₁₋₁₂ alkyl, C₃₋₈ cycloalkyl, linear or branched C₁₋₁₂ hydroxyalkyl, a carboxy group, a sulfonyl group, halogen, and phenyl.

It is to be understood that if not explicitly stated otherwise, all stereoisomers, conformations and configurations are encompassed by compounds and functional groups which can be present as different stereoisomers or in different conformations and configurations. For example, the term “inositol” is to be understood as to include all stereoisomers and conformations such as myo-, scyllo-, muco-, D-chiro-, neo-inositol, L-chiro-, allo-, epi-, and cis-inositol. For example, the term “hexanetriol” is to be understood as to include all hexane isomers including three hydroxyl groups such as 1,1,1-hexanetriol, 1,1,2-hexanetriol, 1,2,2-hexanetriol, 1,2,3-hexanetriol, 1,2,4-hexanetriol, 1,2,5-hexanetriol, 1,2,6-hexanetriol, 1,3,5-hexanetriol, 1,3,6-hexanetriol, 2,3,4-hexanetriol, 2,3,5-hexanetriol etc.

The meanings and preferred meanings described herein for A, R¹, R², and X apply to all compounds and processes including the precursors of the compounds in any of the process steps detailed herein.

As used herein, the term “comprising” is to be construed as encompassing both “including” and “consisting of”, both meanings being specifically intended, and hence individually disclosed, embodiments according to the present invention.

As used herein, the articles “a” and “an” preceding an element or component are intended to be nonrestrictive regarding the number of instances (i.e. occurrences) of the element or component. Therefore, “a” or “an” is to be read to include one or at least one, and the singular word form of the element or component also includes the plural unless the number is obviously meant to be singular.

As used herein, the term “about” modifying the quantity of a substance, ingredient, component, or parameter employed refers to variation in the numerical quantity that can occur, for example, through typical measuring and handling procedures, e.g., liquid handling procedures used for making concentrates or solutions. Furthermore, variation can occur from inadvertent error in measuring procedures, differences in the manufacture, source, or purity of the ingredients employed to carry out the methods, and the like. In one embodiment, the term “about” means within 10% of the reported numerical value. In a more specific embodiment, the term “about” means within 5% of the reported numerical value.

As outlined above, subject of the present invention provides a compound of formula 1

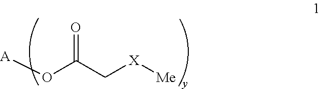

wherein
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A.

In one embodiment, the organic polyol is a linear, branched, or cyclic organic compound with 2 to 18 carbon atoms having at least 2 hydroxyl groups.

In one embodiment, the organic polyol is selected from a linear or branched C₂₋₁₂ alkyl substituted with at least 2 hydroxyl groups or a C₃₋₈cycloalkyl substituted with at least 2 hydroxyl groups.

Preferably, the linear or branched C₂₋₁₂ alkyl substituted with at least 2 hydroxyl groups is selected from the group consisting of ethylene glycol, propanediol, glycerol, propanetriol, trimethylolpropane, pentaerythritol, butanediol, butanetriol, butanetetrol, 2-methyl-propanetriol, pentanediol, pentanetriol, 3-methyl-pentanetriol, pentanetetrol, hexanediol, hexanetriol, hexanetetrol, hexanepentol, and combinations thereof. More preferably, the linear or branched C₂₋₁₂ alkyl substituted with at least 2 hydroxyl groups is 1,3-butanediol or glycerol.

Preferably, the C₃₋₈cycloalkyl substituted with at least 2 hydroxyl groups is selected from the group consisting of cyclobutanediol, cyclopentanediol, cyclopentantriol, cyclopentanetetrol, cyclopentanepentol, cyclohexanediol, cyclohexantriol, cyclohexanetetrol, cyclohexanepentol, cyclohexanehexol, dihydroxytetrahydrofuran, trihydroxytetrahydrofuran, tetrahydroxytetrahydrofuran, dihydroxytetrahydropyrane, trihydroxytetrahydropyrane, tetrahydroxytetrahydropyrane, isosorbide, and combinations thereof.

In one embodiment, the organic polyol is selected from the group consisting of monosaccharides, disaccharides, oligosaccharides, sugar alcohols, and sugar acids.

Monosaccharides generally have the chemical formula CₙH₂ₙOₙ. Monosaccharides can be classified by the number x of carbon atoms they contain (CH₂O)ₓ: trioses (x=3), tetroses (x=4), pentoses (x=5), hexoses (x=6) and heptoses (x=7).

In one embodiment, the monosaccharide is selected from trioses, tetroses, pentoses, hexoses, and heptoses. Preferably, the monosaccharide is selected from aldotrioses, ketotrioses, aldotetroses, ketotetroses, aldopentoses, ketopentoses, aldohexosen, ketohexoses, aldoheptoses and ketoheptoses.

In one embodiment, the monosaccharide is selected from the group consisting of glyceraldehyde, dihydroxyacetone, erythrose, threose, erythrulose, ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Generally, disaccharides comprise at least two units of monosaccharides that are joined by glycosidic linkage. In one embodiment, the disaccharide is selected from the group consisting of sucrose, sucralose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, mannobiose, melibiose, melibiulose, rutinose, rutinulose, xylobiose, and combinations thereof.

Oligosaccharides generally comprise three or more units, typically three to ten units, of monosaccharides. In one embodiment, the oligosaccharide is selected from the group consisting of stevioside, steviol glycoside, raubaudioside A, raubaudioside B, raubaudioside C, raubaudioside D, raffinose, and combinations thereof.

Sugar alcohols (also called polyhydric alcohols, polyalcohols, alditols or glycitols) are organic compounds, typically derived from sugars, containing one hydroxyl group (—OH) attached to each carbon atom.

In one embodiment, the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and combinations thereof.

A sugar acid is generally a monosaccharide with a carboxyl group at one end or both ends of the carbon chain. Main classes of sugar acids include aldonic acids, ulosonic acids, uronic acids, and aldaric acids. In aldonic acids, the aldehyde group (—CHO) located at the initial end (position 1) of an aldose is oxidized. In ulosonic acids, the —CH₂(OH) group at the initial end of a 2-ketose is oxidized yielding an a-ketoacid. In uronic acids, the —CH₂(OH) group at the terminal end of an aldose or ketose is oxidized. In aldaric acids, both ends (—CHO and —CH₂(OH)) of an aldose are oxidized.

In one embodiment, the sugar acid is selected from aldonic acids, ulosonic acids, uronic acids, and aldaric acids. Preferably, the sugar acid is selected from the group consisting of glyceric acid, tartaric acid, xylonic acid, gluconic acid, ascorbic acid, neuraminic acid, ketodeoxyoctonic acid, glucuronic acid, galacturonic acid, iduronic acid, mucic acid, saccharic acid, and combinations thereof.

Preferably, the organic polyol is selected from the group consisting of 1,3-butanediol, glycerol, sorbitol, xylitol, mannitol, erythritol, maltitol, glucose, glucitol, ribulose, pentaerythritol, trimethylolpropane, stevioside, and isosorbide. More preferably, the organic polyol is glycerol or 1,3-butanediol. Even more preferably, the organic polyol is 1,3-butanediol.

In one embodiment, in the compound according to formula 1, y is equal to the number of hydroxyl groups of the initial polyol A.

In one embodiment, the residues

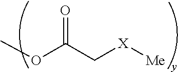

in the compound according to formula 1 may be identical or each independently different for each occurrence.

Preferably, X is —C(OR¹)(OR²)— or —C(SR¹)(SR²)—. More preferably, X is —C(OR¹)(OR²)—.

R¹ and R² together may form a 5 to 8 membered cyclic ketal, a 5 to 8 membered cyclic thioketal, or a 5 to 8 membered 1,3-oxathiolane. Preferably, R¹ and R² together form a 5 to 8 membered cyclic ketal, a 5 to 8 membered cyclic thioketal, or a 5 to 8 membered 1,3-oxathiolane. Preferably, R¹ and R² together form a 5 to 8 membered cyclic ketal. Even more preferably, R¹ and R² together form a 6-membered cyclic ketal.

In one embodiment, X is —C(OR¹)(OR²)— and preferably forms a 6-membered cyclic ketal derived from 1,3-butanediol with X having the following structure

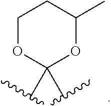 [SMILES: *C1(*)OCCC(C)O1]

In one embodiment, X is —C(SR¹)(SR²)— and preferably forms a 6-membered cyclic thioketal derived from 1,3-butanedithiol with X having the following structure

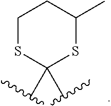 [SMILES: *C1(*)SCCC(C)S1]

In one embodiment, X is —C(OR¹)(SR²)— or —C(SR¹)(OR²)— and preferably forms a 6-membered cyclic 1,3-oxathiolane derived from 1-mercaptobutane-3-ol or 3-mercaptobutane-1-ol with X having the following structure

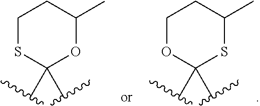

Preferably, the compound according to formula 1 is

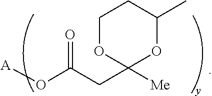

wherein A is derived from an organic polyol with at least 2 hydroxyl groups, and
y is from 2 to the number of hydroxyl groups of the initial organic polyol A.

In one embodiment, the compound according to formula 1 is selected from the group consisting of

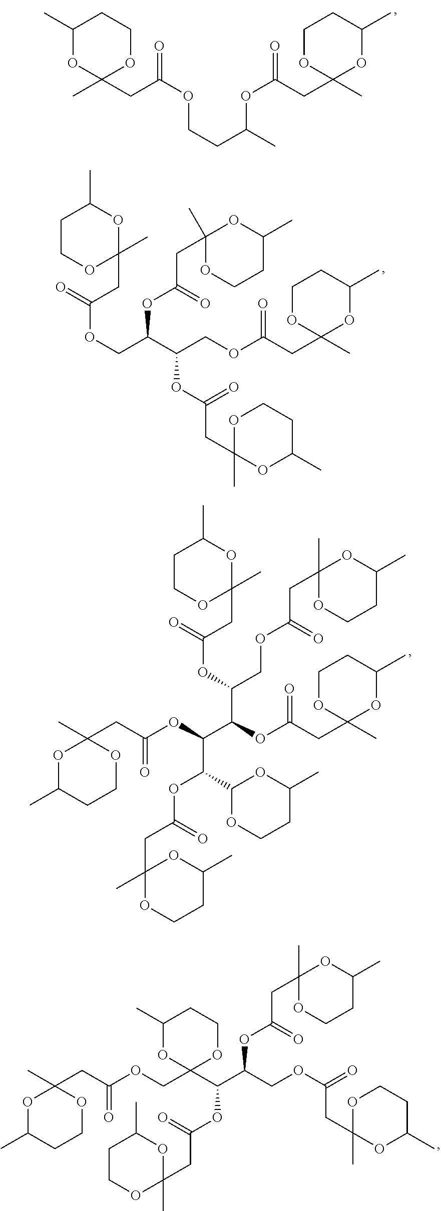

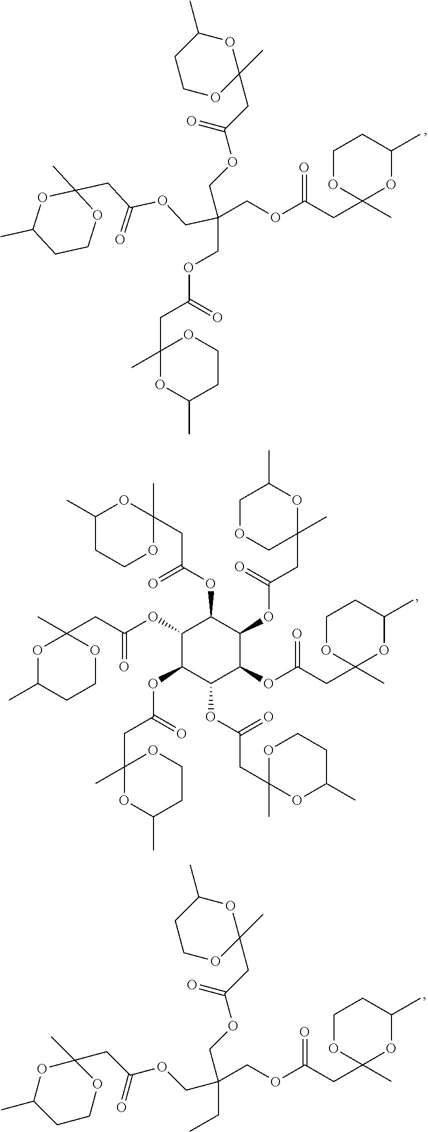

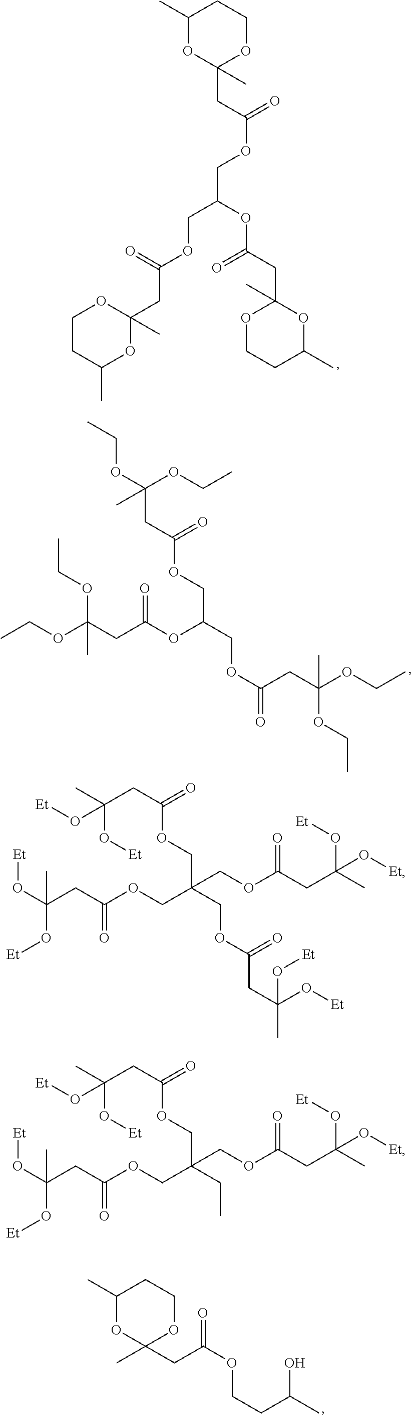

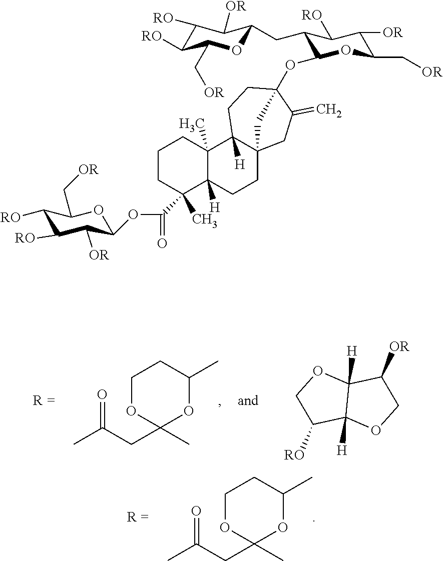

In another aspect, the present invention provides a process for the preparation of a compound of formula 1

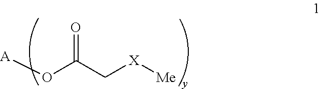

wherein
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A;
wherein the process comprises:
   (i) reacting an organic polyol of formula 2 with diketene 3 resulting in the formation of a compound according to formula 4;

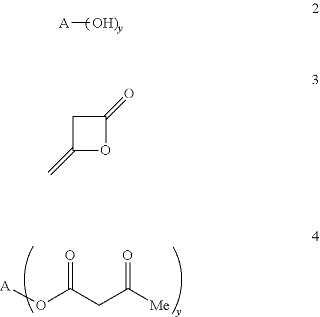

and
(ii) reacting the compound of formula 4 with an alcohol or a thiol resulting in the formation of a compound according to formulas 5 to 8

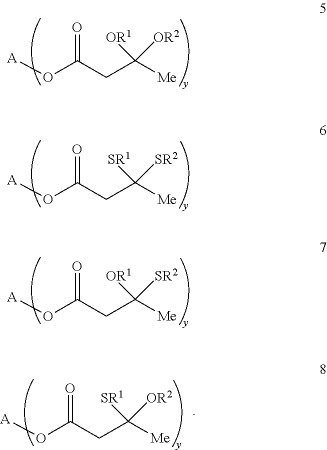

All embodiments and preferred embodiments disclosed above with respect to the compound of formula 1 likewise apply for the process of preparing a compound of formula 1.

The inventors surprisingly found that the process according to the present invention for the preparation of compounds according to formula 1 achieves significantly improved atom economy and cost efficiency if a compound according to formula 2 is reacted with diketene 3 resulting in the formation of a compound according to formula 4. More BHB units or BHB derivate units per polyol core is favorable for applications in which a high ratio of or BHB units or derivatives thereof to the polyol is desired. Moreover, the terminal acetoacetate units are further reacted to ketals, thioketals, or 1,3-oxathiolanes to provide protected BHB units. The process according to the present invention achieves a high BHB unit content per polyol unit. Ketalization of the terminal acetoacetate units with the BHB derivative 1,3-butanediol further increases the amount of BHB derivatives per polyol unit.

In one embodiment, reaction step (i) is performed in the presence of an organic amine catalyst. Suitable organic amine catalysts include tertiary amines. Preferably, the organic amine catalyst is 1,4-diazabicyclo[2.2.2]octane (DABCO).

Depending on the type of the organic polyol, the process for the preparation of a compound of formula 1 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 1 is performed without a solvent. In another embodiment, the process for the preparation of a compound of formula 1 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan, or o/m/p-xylene. Preferably, the organic solvent is ethyl acetate.

In one embodiment, in the process for the preparation of a compound of formula 1, reaction step (i) is performed at temperature of 20-100° C. Preferably, reaction step (i) is performed at temperature of 40-70° C. Additionally, the reaction temperature of reaction step (i) may be maintained at 40-70° C. after complete addition of diketene 3.

In one embodiment, during reaction step (i) diketene 3 is slowly added over a period of 1-6 h, e.g. dropwise, to the reaction mixture, to avoid the formation of side products.

After step (i), the compound of formula 4 is then reacted with an alcohol, a thiol, or a mercapto alcohol resulting in the formation of a compound according to formula 5 to 8.

Preferably, the compound of formula 4 is reacted with a diol, dithiol, or mercapto alcohol to yield a 5 to 8 membered cyclic ketal, a 5 to 8 membered cyclic thioketal, or a 5 to 8 membered 1,3-oxathiolane. The ketal, thioketal or 1,3-oxathiolane is formed by condensation of the keto function of one or more acetoacetate units of the compound of formula 4 with the diol, dithiol, or mercapto alcohol. In this case, in compounds according to formula 5 to 8, R¹ and R² together form a 5 to 8 membered cyclic ketal, a 5 to 8 membered cyclic thioketal, or a 5 to 8 membered 1,3-oxathiolane.

In one embodiment, the diol is selected from the group consisting of ethylenglycol, 1,2-propanediol, 1,2-dimethyl-1,2-propanediol, 1,3-propanediol, 2-methyl-1,3-propanediol, 1,3-butanediol, 2-methyl-1,3-butanediol, 2,2-dimethyl-1,3-butanediol, 1,2-dimethyl-1,3-butanediol, 2,3-butanediol, 2-methyl-2,3-butanediol, pinacol, 1,4-butanediol, 1,5-pentanediol, and combinations thereof. Preferably, the diol is 1,3-butanediol.

In one embodiment, the dithiol is selected from the group consisting of ethane-1,2-dithiol, 1,2-propandithiol, 1,2-dimethyl-1,2-propandithiol, 1,3-propandithiol, 2-methyl-1,3-propandithiol, 1,3-butanedithiol, 2-methyl-1,3-butanedithiol, 2,2-dimethyl-1,3-butanedithiol, 1,2-dimethyl-1,3-butanedithiol, 2,3-butanedithiol, 2-methyl-2,3-butanedithiol, 2,3-dimethyl-2,3-butanedithiol, 1,4-butanedithiol, 1,5-pentanedithiol, and combinations thereof. Preferably, the dithiol is 1,3-butanedithiol.

In one embodiment, the mercapto alcohol is selected from the group consisting of mercaptoethanol, 3-mercapto-1-propanol, 1-mercaptopropane-2-ol, 2-mercaptopropane-1-ol, 3-mercapto-3-methylbutane-2-ol, 3-mercapto-2-methylbutane-2-ol, 1,3-propanedithiol, 3-mercapto-2-methylpropane-1-ol, 3-mercapto-2,2-dimethylpropane-1-ol, 4-mercaptobutane-2-ol, 1-mercaptobutane-3-ol, 3-mercaptobutane-1-ol, 3-mercapto-2-methylbutane-1-ol, 4-mercapto-3-methylbutane-2-ol, 4-mercapto-3,3-dimethylbutane-2-ol, 3-mercapto-2,2-dimethylbutane-1-ol, 4-mercapto-3-methylpentane-2-ol, 3-mercaptobutane-2-ol, 3-mercapto-2-methylbutane-2-ol, 3-mercapto-3-methylbutane-2-ol, 3-mercapto-2,3-dimethylbutane-2-ol, 4-mercaptobutane-1-ol, and 5-mercaptopentane-1-ol. Preferably, the mercapto alcohol is 1-mercaptobutane-3-ol or 3-mercaptobutane-1-ol.

In one embodiment, in the compound according to formula 5, R¹ and R² together form a 6-membered cyclic ketal formed by condensation of the keto function of one or more acetoacetate units of the compound of formula 4 with 1,3-butanediol

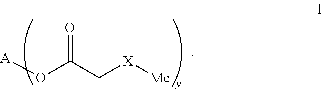

In another aspect, the present invention provides a compound of formula 9

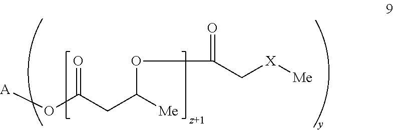

wherein
z is 0 or more,
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A.

In one embodiment, z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20. In one embodiment, z is from 0-20 such as 0, 1, 2, 3, 4, 5, 6, 7, 8, 9, 10, 11, 12, 13, 14, 15, 16, 17, 18, 19, or 20. In one embodiment, z is from 0-20, such as 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 1, or 0. Preferably, z is 0 or 1.

In one embodiment, the organic polyol is a linear, branched, or cyclic organic compound with 2 to 18 carbon atoms having at least 2 hydroxyl groups.

In one embodiment, the organic polyol is selected from a linear or branched C₂₋₁₂ alkyl substituted with at least 2 hydroxyl groups or a C₃₋₈cycloalkyl substituted with at least 2 hydroxyl groups.

Preferably, the linear or branched C₂₋₁₂ alkyl substituted with at least 2 hydroxyl groups is selected from the group consisting of ethylene glycol, propanediol, glycerol, propanetriol, trimethylolpropane, pentaerythritol, butanediol, butanetriol, butanetetrol, 2-methyl-propanetriol, pentanediol, pentanetriol, 3-methyl-pentanetriol, pentanetetrol, hexanediol, hexanetriol, hexanetetrol, hexanepentol, and combinations thereof. More preferably, the linear or branched C₂₋₁₂ alkyl substituted with at least 2 hydroxyl groups is 1,3-butanediol or glycerol.

Preferably, the C₃₋₈cycloalkyl substituted with at least 2 hydroxyl groups is selected from the group consisting of cyclobutanediol, cyclopentanediol, cyclopentantriol, cyclopentanetetrol, cyclopentanepentol, cyclohexanediol, cyclohexanetriol, cyclohexanetetrol, cyclohexanepentol, cyclohexanehexol, dihydroxytetrahydrofuran, trihydroxytetrahydrofuran, tetrahydroxytetrahydrofuran, dihydroxytetrahydropyrane, trihydroxytetrahydropyrane, tetrahydroxytetrahydropyrane, isosorbide, and combinations thereof.

In one embodiment, the organic polyol is selected from the group consisting of monosaccharides, disaccharides, oligosaccharides, sugar alcohols, and sugar acids.

In one embodiment, the monosaccharide is selected from trioses, tetroses, pentoses, hexoses, and heptoses. Preferably, the monosaccharide is selected from aldotrioses, ketotrioses, aldotetroses, ketotetroses, aldopentoses, ketopentoses, aldohexosen, ketohexoses, aldoheptoses and ketoheptoses.

Preferably, the monosaccharide is selected from the group consisting of glyceraldehyde, dihydroxyacetone, erythrose, threose, erythrulose, ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Preferably, the disaccharide is selected from the group consisting of sucrose, sucralose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, mannobiose, melibiose, melibiulose, rutinose, rutinulose, xylobiose, and combinations thereof.

Preferably, the oligosaccaride is selected from the group consisting of stevioside, steviol glycoside, raubaudioside A, raubaudioside B, raubaudioside C, raubaudioside D, raffinose, and combinations thereof.

Preferably, the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and combinations thereof.

In one embodiment, the sugar acid is selected from aldonic acids, ulosonic acids, uronic acids, and aldaric acids. Preferably, the sugar acid is selected from the group consisting of glyceric acid, tartaric acid, xylonic acid, gluconic acid, ascorbic acid, neuraminic acid, ketodeoxyoctonic acid, glucuronic acid, galacturonic acid, iduronic acid, mucic acid, saccharic acid, and combinations thereof.

Preferably, the organic polyol is selected from the group consisting of 1,3-butanediol, glycerol, sorbitol, xylitol, mannitol, erythritol, maltitol, glucose, glucitol, ribulose, pentaerythritol, trimethylolpropane, stevioside, and isosorbide. More preferably, the organic polyol is glycerol or 1,3-butanediol. Even more preferably, the organic polyol is 1,3-butanediol.

In one embodiment, in the compound according to formula 9, y is equal to the number of hydroxyl groups of the initial polyol A.

In one embodiment, the residues

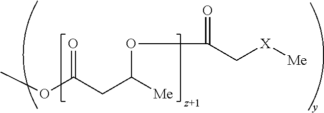

in the compound according to formula 9 may be identical or each independently different for each occurrence.

In one embodiment, the compound according to formula 9, all p-hydroxyl butyric acid ester units are either D-configured or L-configured. In another embodiment, all p-hydroxyl butyric acid ester units are present in the compound according to formula 9 as a non-racemic mixture of D- and L-configurations.

Preferably, X is —C(OR¹)(OR²)— or —C(SR¹)(SR²)—. More preferably, X is —C(OR¹)(OR²)—.

R¹ and R² together may form a 5 to 8 membered cyclic ketal, a 5 to 8 membered cyclic thioketal, or a 5 to 8 membered 1,3-oxathiolane. Preferably, R¹ and R² together form a 5 to 8 membered cyclic ketal, a 5 to 8 membered cyclic thioketal, or a 5 to 8 membered 1,3-oxathiolane. Preferably, R¹ and R² together form a 5 to 8 membered cyclic ketal. Even more preferably, R¹ and R² together form a 6-membered cyclic ketal.

In one embodiment, X is —C(OR¹)(OR²)— and preferably forms a 6-membered cyclic ketal derived from 1,3-butanediol with X having the following structure

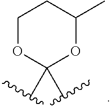 [SMILES: *C1(*)OCCC(C)O1]

In one embodiment, X is —C(SR¹)(SR²)— and preferably forms a 6-membered cyclic thioketal derived from 1,3-butanedithiol with X having the following structure

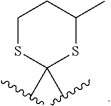 [SMILES: *C1(*)SCCC(C)S1]

In one embodiment, X is —C(OR¹)(SR²)— or —C(SR¹)(OR²)— and preferably forms a 6-membered cyclic 1,3-oxathiolane derived from 1-mercaptobutane-3-ol or 3-mercaptobutane-1-ol with X having the following structure

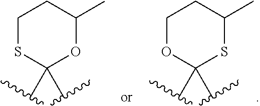

Preferably, the compound according to formula 9 is

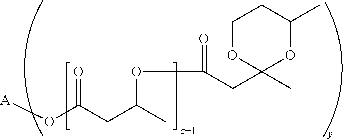

wherein
z is 0 or more, preferably 0-5,
A is derived from an organic polyol with at least 2 hydroxyl groups, and
y is from 2 to the number of hydroxyl groups of the initial organic polyol A.

In one embodiment, the compound according to formula 9 is

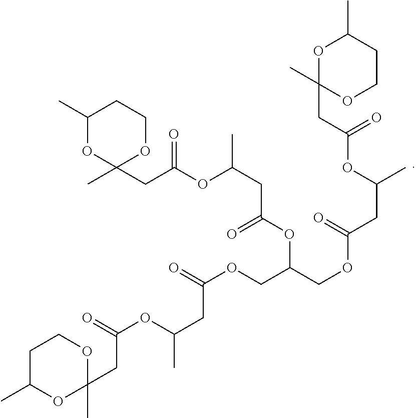 [SMILES: CC(CC(=O)OCC(COC(=O)CC(C)OC(=O)CC1(C)OCCC(C)O1)OC(=O)CC(C)OC(=O)CC1(C)OCCC(C)O1)OC(=O)CC1(C)OCCC(C)O1]

In another aspect, the present invention provides a process for the preparation of a compound of formula

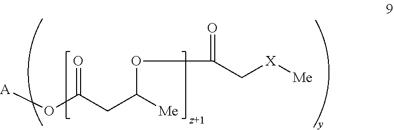

wherein
z is 0 or more,
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal or a 5 to 8 membered cyclic thioketal, and
y is from 2 to the number of hydroxyl groups of the initial organic polyol A;
wherein the process comprises:
   (i) reacting a compound of formula 10 with diketene 3 resulting in the formation of a compound according to formula 11;

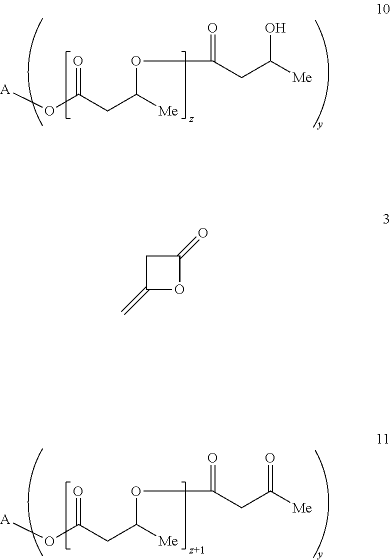

and
(ii) reacting the compound of formula 11 with an alcohol or a thiol resulting in the formation of a compound according to formula 12 to 15

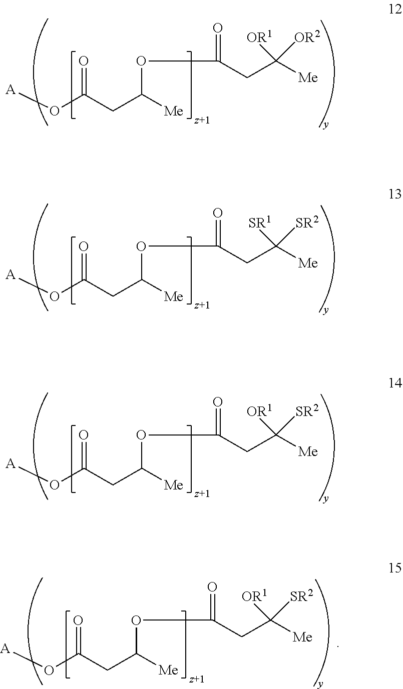

All embodiments and preferred embodiments disclosed above with respect to the compound of formula 9 likewise apply for the process of preparing a compound of formula 9.

The inventors surprisingly found that the process according to the present invention for the preparation of a compound of formula 9 achieves significantly improved atom economy and cost efficiency if a compound according to formula 10 is reacted with diketene 3 resulting in the formation of a compound according to formula 11. More acetoacetate and/or BHB units per polyol core is favorable for applications in which a high ratio of acetoacetate and/or BHB units or derivatives thereof to the polyol is desired. Moreover, the inventors surprisingly found that after hydrogenation of the terminal acetoacetate function in compound 11, the process of reacting the obtained compound with diketene 3 according to step (i) may be repeated to increase the number of “z” BHB units. This ultimately yields dendrimers with multiple BHB units of a desired length. The terminal acetoacetate units are then further reacted to ketals, thioketals, or 1,3-oxathiolanes to provide protected BHB units. Thus, the process according to the present invention achieves a high BHB unit content per polyol unit. Ketalization of the terminal acetoacetate units with the BHB derivative 1,3-butanediol further increases the amount of BHB derivatives per polyol unit.

In one embodiment, reaction step (i) is performed in the presence of an organic amine catalyst. Suitable organic amine catalysts include tertiary amines. Preferably, the organic amine catalyst is 1,4-diazabicyclo[2.2.2]octane (DABCO).

In one embodiment, prior to reaction step (ii), the compound of formula 11 is reacted with hydrogen in the presence of a catalyst resulting in the hydrogenation of the terminal acetoacetate function in the compound of formula 11 to yield terminal BHB groups. In this case, step (i) is repeated with the resulted hydrogenated compound of formula 11 to increase the number z by 1. This may be repeated any number of times until dendrimers with multiple BHB units of a desired length are obtained. In one embodiment, hydrogenation and step (i) are repeated from 1 to 100 times such as 1 to 50 time, 1 to 40 times, 1 to 30 time, or 1 to 25 times, such as 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, or 25 times.

When compound of formula 11 is reacted with hydrogen, this may be done in the presence of a catalyst. In one embodiment, hydrogenation is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or a Rh-based catalyst.

In one embodiment, when a metal-based catalyst is used, hydrogenation is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino)butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C₃-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

By using a chiral ligand, the configuration of the β-hydroxyl butyric acid ester units in the compound according to formula 9 may be controlled. In one embodiment, the compound according to formula 9, all β-hydroxyl butyric acid ester units are either D-configured or L-configured. In another embodiment, all β-hydroxyl butyric acid ester units are present in the compound according to formula 9 as a non-racemic mixture of D- and L-configurations.

Preferably, hydrogenation is performed in the presence of a Ru-based catalyst. A preferred Ru-based catalyst is a Ruthenium oxide catalyst such as RuO₂. Further preferred Ru-based catalysts include Ru(OAc)₂(BINAP) and Ru(Cl)₂(BINAP).

Hydrogenation may be performed in a closed vessel under hydrogen pressure. Preferably, hydrogenation is performed at 5-30 bar hydrogen pressure and even more preferably at 10-20 bar hydrogen pressure.

In one embodiment, hydrogenation is performed at a temperature of 30-90° C. Preferably, hydrogenation is performed at a temperature of 50-70° C. and more preferably, hydrogenation is performed at a temperature of about 60° C.

In one embodiment, during hydrogenation the reaction mixture is stirred at 800-1200 rpm so as to ensure sufficient hydrogen diffusion into the reaction mixture.

Depending on the type of the organic polyol, the process for the preparation of a compound of formula 9 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 9 is performed without a solvent. In another embodiment, the process for the preparation of a compound of formula 9 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan, methanol, ethanol, or o/m/p-xylene. Preferably, the organic solvent is ethyl acetate.

In one embodiment, in the process for the preparation of a compound of formula 9, reaction step (i) is performed at temperature of 20-100° C. Preferably, reaction step (i) is performed at temperature of 40-70° C. Additionally, the reaction temperature of reaction step (i) may be maintained at 40-70° C. after complete addition of diketene 3.

In one embodiment, during reaction step (i) diketene 3 is slowly added over a period of 1-6 h, e.g. dropwise, to the reaction mixture, to avoid the formation of side products.

After step (i), the compound of formula 11 is then reacted with an alcohol, a thiol, or a mercapto alcohol resulting in the formation of a compound according to formula 12 to 15.

Preferably, the compound of formula 11 is reacted with a diol, dithiol or mercapto alcohol to yield a 5 to 8 membered cyclic ketal, a 5 to 8 membered cyclic thioketal, or a 5 to 8 membered 1,3-oxathiolane. The ketal, thioketal, or 1,3-oxathiolane is formed by condensation of the keto function of one or more acetoacetate units of the compound of formula 11 with the diol, dithiol, or mercapto alcohol. In this case, in compounds according to formula 12 to 15, R¹ and R² together form a 5 to 8 membered cyclic ketal, a 5 to 8 membered cyclic thioketal, or a 5 to 8 membered 1,3-oxathiolane.

In one embodiment, the diol is selected from the group consisting of ethylenglycol, 1,2-propanediol, 1,2-dimethyl-1,2-propanediol, 1,3-propanediol, 2-methyl-1,3-propanediol, 1,3-butanediol, 2-methyl-1,3-butanediol, 2,2-dimethyl-1,3-butanediol, 1,2-dimethyl-1,3-butanediol, 2,3-butanediol, 2-methyl-2,3-butanediol, pinacol, 1,4-butanediol, 1,5-pentanediol, and combinations thereof. Preferably, the diol is 1,3-butanediol.

In one embodiment, the dithiol is selected from the group consisting of ethane-1,2-dithiol, 1,2-propanedithiol, 1,2-dimethyl-1,2-propanedithiol, 1,3-propanedithiol, 2-methyl-1,3-propanedithiol, 1,3-butanedithiol, 2-methyl-1,3-butanedithiol, 2,2-dimethyl-1,3-butanedithiol, 1,2-dimethyl-1,3-butanedithiol, 2,3-butanedithiol, 2-methyl-2,3-butanedithiol, 2,3-dimethyl-2,3-butanedithiol, 1,4-butanedithiol, 1,5-pentanedithiol, and combinations thereof. Preferably, the dithiol is 1,3-butanedithiol.

In one embodiment, the dithiol is selected from the group consisting of mercaptoethanol, 3-mercapto-1-propanol, 1-mercaptopropane-2-ol, 2-mercaptopropane-1-ol, 3-mercapto-3-methylbutane-2-ol, 3-mercapto-2-methylbutane-2-ol, 1,3-propanedithiol, 3-mercapto-2-methylpropane-1-ol, 3-mercapto-2,2-dimethylpropane-1-ol, 4-mercaptobutane-2-ol, 1-mercaptobutane-3-ol, 3-mercaptobutane-1-ol, 3-mercapto-2-methylbutane-1-ol, 4-mercapto-3-methylbutane-2-ol, 4-mercapto-3,3-dimethylbutane-2-ol, 3-mercapto-2,2-dimethylbutane-1-ol, 4-mercapto-3-methylpentane-2-ol, 3-mercaptobutane-2-ol, 3-mercapto-2-methylbutane-2-ol, 3-mercapto-3-methylbutane-2-ol, 3-mercapto-2,3-dimethylbutane-2-ol, 4-mercaptobutane-1-ol, and 5-mercaptopentane-1-ol. Preferably, the mercapto alcohol is 1-mercaptobutane-3-ol or 3-mercaptobutane-1-ol.

In one embodiment, in compounds according to formula 12 to 15, R¹ and R² together form a 6-membered cyclic ketal formed by condensation of the keto function of one or more acetoacetate units of the compound of formula 11 with 1,3-butanediol

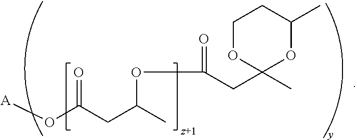

The invention is further defined by the following numbered items:

1. A compound of formula 1

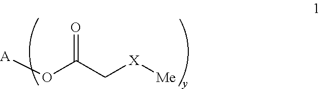

wherein
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A. 2. A compound of formula 9

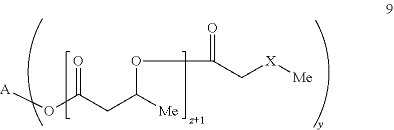

wherein
z is 0 or more,
A is derived from an organic polyol with at least 2 hydroxyl groups,
X is —C(OR¹)(OR²)—, —C(SR¹)(SR²)—, —C(SR¹)(OR²)—, or —C(OR¹)(SR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial polyol A.

3. The compound according to item 2, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.

4. The compound according to any one of items 1 to 3, wherein the organic polyol is selected from a linear or branched C₂₋₁₂ alkyl substituted with at least 2 hydroxyl groups or a C₃₋₈ cycloalkyl substituted with at least 2 hydroxyl groups.

5. The compound according to item 4, wherein the linear or branched C₂₋₁₂ alkyl substituted with at least 2 hydroxyl groups is selected from the group consisting of ethylene glycol, propanediol, glycerol, propanetriol, trimethylolpropane, 1,3-butanediol, butanediol, butanetriol, 2-methyl-propanetriol, pentanediol, pentanetriol, 3-methyl-pentanetriol hexanediol, hexanetriol, pentaerythritol, butanetetrol, pentanetetrol, hexanetetrol, hexanepentol, and combinations thereof,

6. The compound according to item 4 or 5, wherein the linear or branched C₂₋₁₂ alkyl substituted with at least 2 hydroxyl groups is selected from the group consisting of glycerol and 1,3-butanediol, preferably wherein the linear or branched C₂₋₁₂ alkyl substituted with at least 2 hydroxyl groups is 1,3-butanediol.

7. The compound according to any one of items 4 to 6, wherein the C₃₋₈cycloalkyl substituted with at least 2 hydroxyl groups is selected from the group consisting of cyclobutanediol, cyclopentanediol, cyclohexanediol, cyclopentanetriol, cyclohexanetriol, cyclopentanetetrol, cyclohexanetetrol, cyclopentanpentol, cyclohexanpentol, cyclohexanhexol, dihydroxytetrahydrofuran, trihydroxytetrahydrofuran, tetrahydroxytetrahydrofuran, dihydroxytetrahydropyrane, trihydroxytetrahydropyrane, tetrahydroxytetrahydropyrane, isosorbide, and combinations thereof.

8. The compound according to any one of items 1 to 3, wherein the organic polyol is selected from the group consisting of monosaccharides, disaccharides, oligosaccharides, sugar alcohols, and sugar acids.

9. The compound according to item 8, wherein the monosaccharide is selected from trioses, tetroses, pentoses, hexoses, and heptoses, preferably wherein the monosaccharide is selected from aldotrioses, ketotrioses, aldotetroses, ketotetroses, aldopentoses, ketopentoses, aldohexosen, ketohexoses, aldoheptoses and ketoheptoses.

10. The compound according to item 8 or 9, wherein the monosaccharide is selected from the group consisting of glyceraldehyde, dihydroxyacetone, erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

11. The compound according to any one of items 8 to 10, wherein the disaccharide is selected from the group consisting of sucrose, sucralose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, mannobiose, melibiose, melibiulose, rutinose, rutinulose, xylobiose, and combinations thereof.

12. The compound according to any one of items 8 to 11, wherein the oligosaccaride is selected from the group consisting of stevioside, steviol glycoside, raubaudioside A, raubaudioside B, raubaudioside C, raubaudioside D, raffinose, and combinations thereof.

13. The compound according to any one of items 8 to 12, wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and combinations thereof.

14. The compound according to any one of items 8 to 13, wherein the sugar acid is selected from the group consisting of glyceric acid, tartaric acid, xylonic acid, gluconic acid, ascorbic acid, neuraminic acid, ketodeoxyoctonic acid, glucuronic acid, galacturonic acid, iduronic acid, mucic acid, saccharic acid, and combinations thereof.

15. The compound according to any one of items 1 to 14, wherein the organic polyol is selected from the group consisting of 1,3-butanediol, glycerol, sorbitol, xylitol, mannitol, erythritol, maltitol, glucose, glucitol, ribulose, pentaerythritol, trimethylolpropane, stevioside, and isosorbide, preferably wherein the organic polyol is glycerol or 1,3-butanediol, even more preferably wherein the organic polyol is 1,3-butanediol.

16. The compound according to any one of items 1 to 15, wherein y is equal to the number of hydroxyl groups of the initial polyol A.

17. The compound according to any one of items 1 to 16, wherein X is selected from

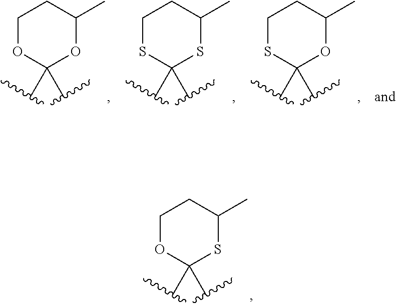

preferably wherein X is

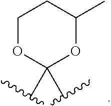 [SMILES: *C1(*)OCCC(C)O1]

18. The compound according to any one of items 1 and 4 to 17, wherein the compound is selected from the group consisting of

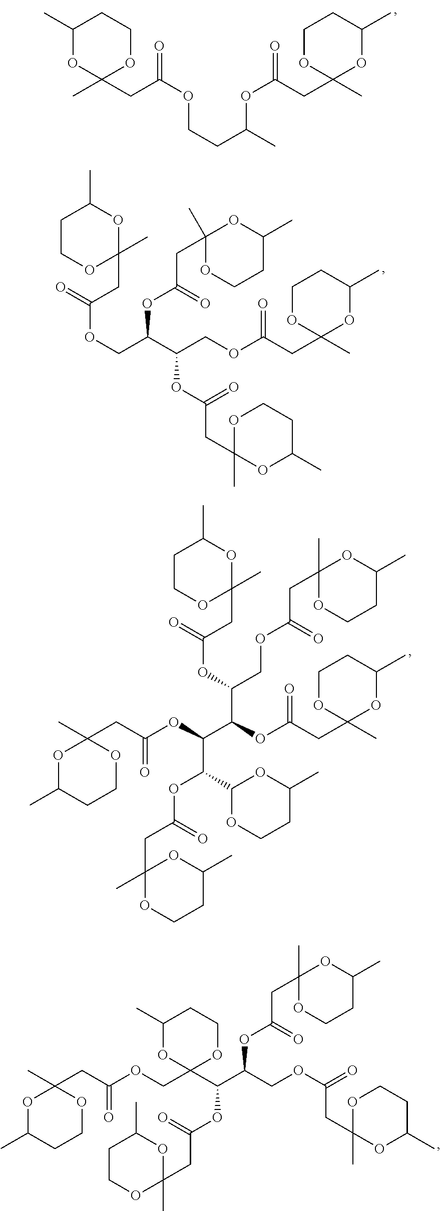

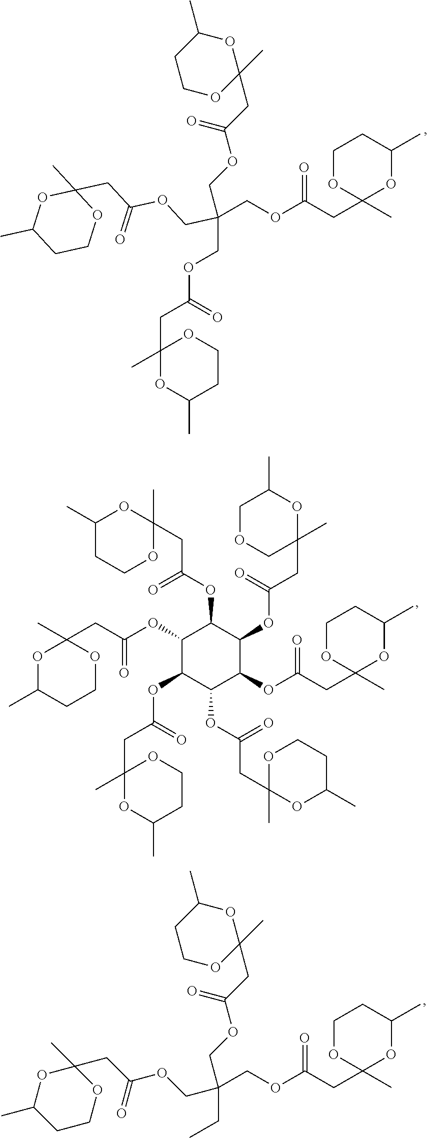

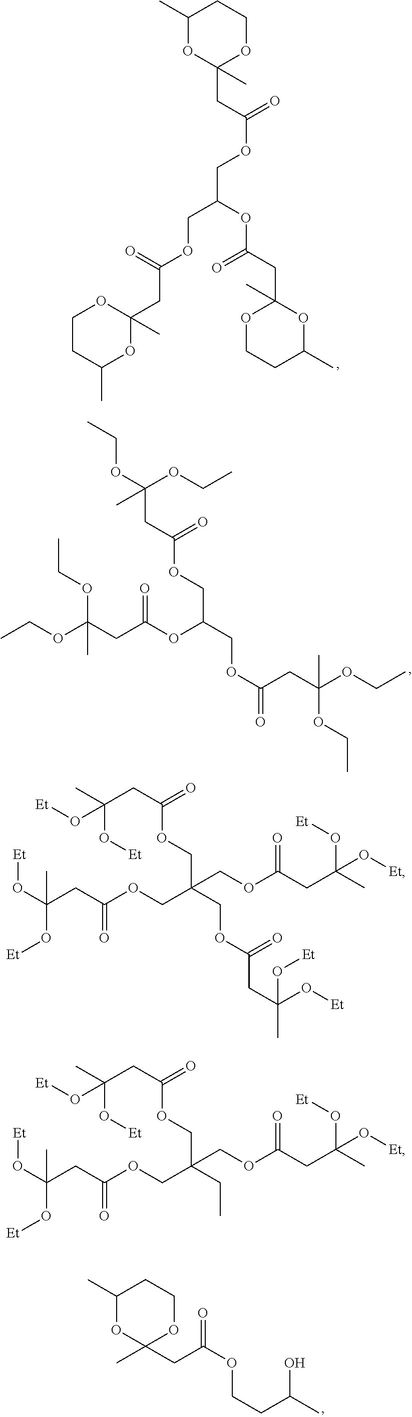

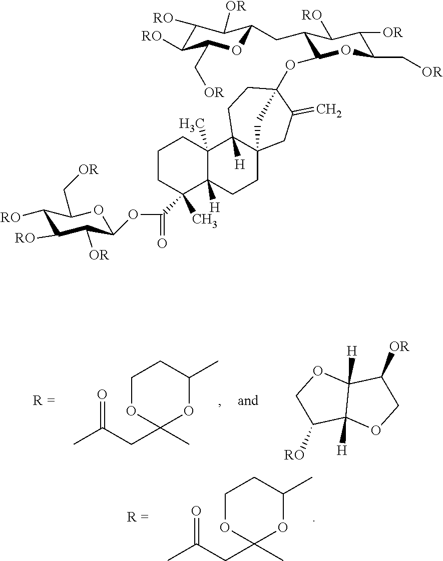

19. The compound according any one of items 2 to 17, wherein the compound is selected from the group consisting of

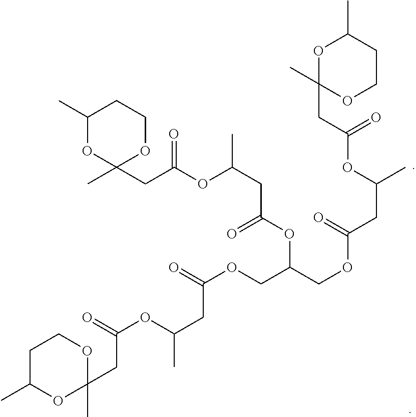 [SMILES: CC(CC(=O)OCC(COC(=O)CC(C)OC(=O)CC1(C)OCCC(C)O1)OC(=O)CC(C)OC(=O)CC1(C)OCCC(C)O1)OC(=O)CC1(C)OCCC(C)O1]

20. A process for the preparation of a compound of formula 1

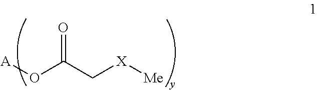

wherein
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A;
wherein the process comprises:
   (i) reacting an organic polyol of formula 2 with diketene 3 resulting in the formation of a compound according to formula 4;

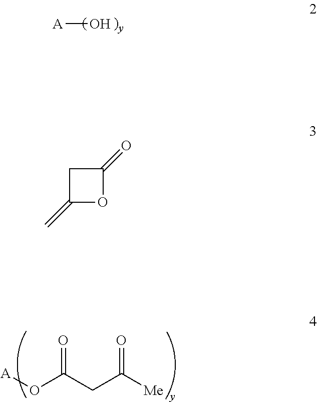

and
(ii) reacting the compound of formula 4 with an alcohol, a thiol, or a mercapto alcohol resulting in the formation of a compound according to formulas 5 to 8

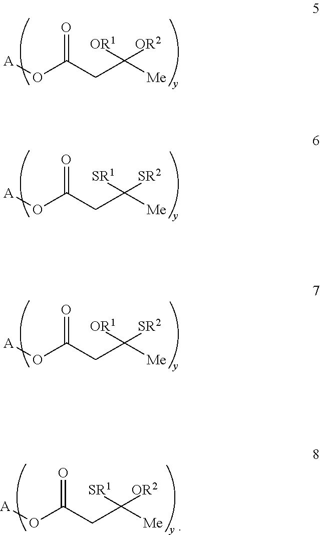

21. A process for the preparation of a compound of formula 9

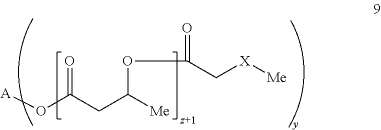

wherein
z is 0 or more,
A is derived from an organic polyol with at least 2 hydroxyl groups,
X is —C(OR¹)(OR²)—, —C(SR¹)(SR²)—, —C(OR¹)(SR²)—, or —C(SR¹)(OR²)—,
R¹ and R² are each independently from each other selected from linear or branched C₁₋₁₂ alkyl, C₃₋₈ cycloalkyl, linear or branched C₁₋₁₂ hydroxyalkyl, and phenyl, or
R¹ and R² may together form a 5 to 8 membered cyclic ketal, a 5 to 8 membered cyclic thioketal, or a 5 to 8 membered 1,3-oxathiolane, and
y is from 2 to the number of hydroxyl groups of the initial organic polyol A;
wherein the process comprises:
   (i) reacting a compound of formula 10 with diketene 3 resulting in the formation of a compound according to formula 11;

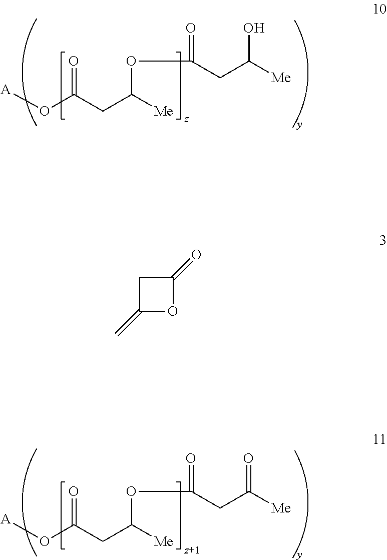

and
(ii) reacting the compound of formula 11 with an alcohol, a thiol, or a mercapto alcohol resulting in the formation of a compound according to formulas 12 to 15

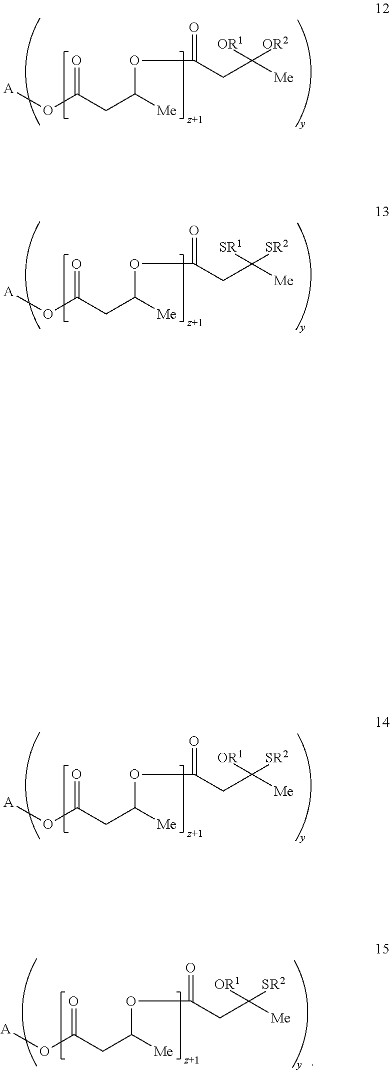

22. The process according to item 21, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.

23. The process according to any one of items 20 to 22, wherein the organic polyol is selected from a linear or branched C₂₋₁₂ alkyl substituted with at least 2 hydroxyl groups or a C₃₋₈ cycloalkyl substituted with at least 2 hydroxyl groups.

24. The process according to item 23, wherein the linear or branched C₂₋₁₂ alkyl substituted with at least 2 hydroxyl groups is selected from the group consisting of ethylene glycol, propanediol, glycerol, propanetriol, trimethylolpropane, 1,3-butanediol, butanediol, butanetriol, 2-methyl-propanetriol, pentanediol, pentanetriol, 3-methyl-pentanetriol hexanediol, hexanetriol, pentaerythritol, butanetetrol, pentanetetrol, hexanetetrol, hexanepentol, and combinations thereof,

25. The process according to item 23 or 24, wherein the linear or branched C₂₋₁₂ alkyl substituted with at least 2 hydroxyl groups is selected from the group consisting of glycerol and 1,3-butanediol, preferably wherein the linear or branched C₂₋₁₂ alkyl substituted with at least 2 hydroxyl groups is 1,3-butanediol.

26. The process according to any one of items 23 to 25, wherein the C₃₋₈ cycloalkyl substituted with at least 2 hydroxyl groups is selected from the group consisting of cyclobutanediol, cyclopentanediol, cyclohexanediol, cyclopentanetriol, cyclohexanetriol, cyclopentanetetrol, cyclohexanetetrol, cyclopentanpentol, cyclohexanpentol, cyclohexanhexol, dihydroxytetrahydrofuran, trihydroxytetrahydrofuran, tetrahydroxytetrahydrofuran, dihydroxytetrahydropyrane, trihydroxytetrahydropyrane, tetrahydroxytetrahydropyrane, isosorbide, and combinations thereof.

27. The process according to any one of items 20 to 22, wherein the organic polyol is selected from the group consisting of monosaccharides, disaccharides, oligosaccharides, sugar alcohols, and sugar acids.

28. The process according to item 27, wherein the monosaccharide is selected from trioses, tetroses, pentoses, hexoses, and heptoses, preferably wherein the monosaccharide is selected from aldotrioses, ketotrioses, aldotetroses, ketotetroses, aldopentoses, ketopentoses, aldohexosen, ketohexoses, aldoheptoses and ketoheptoses.

29. The process according to item 27 or 28, wherein the monosaccharide is selected from the group consisting of glyceraldehyde, dihydroxyacetone, erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

30. The process according to any one of items 27 to 29, wherein the disaccharide is selected from the group consisting of sucrose, sucralose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, mannobiose, melibiose, melibiulose, rutinose, rutinulose, xylobiose, and combinations thereof.

31. The process according to any one of items 27 to 30, wherein the oligosaccaride is selected from the group consisting of stevioside, steviol glycoside, raubaudioside A, raubaudioside B, raubaudioside C, raubaudioside D, raffinose, and combinations thereof.

32. The process according to any one of items 27 to 31, wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and combinations thereof.

33. The process according to any one of items 27 to 32, wherein the sugar acid is selected from the group consisting of glyceric acid, tartaric acid, xylonic acid, gluconic acid, ascorbic acid, neuraminic acid, ketodeoxyoctonic acid, glucuronic acid, galacturonic acid, iduronic acid, mucic acid, saccharic acid, and combinations thereof.

34. The process according to any one of items 20 to 33, wherein the organic polyol is selected from the group consisting of 1,3-butanediol, glycerol, sorbitol, xylitol, mannitol, erythritol, maltitol, glucose, glucitol, ribulose, pentaerythritol, trimethylolpropane, stevioside, and isosorbide, preferably wherein the organic polyol is glycerol or 1,3-butanediol, even more preferably wherein the organic polyol is 1,3-butanediol.

35. The process according to any one of items 20 to 34, wherein y is equal to the number of hydroxyl groups of the initial polyol A.

36. The process according to any one of items 20 to 35, wherein the compound of formula 4 or the compound of formula 11 is reacted in step (ii) with a diol, dithiol, or mercapto alcohol to yield a 5 to 8 membered cyclic ketal, a 5 to 8 membered cyclic thioketal, or a 5 to 8 membered 1,3-oxathiolane.

37. The process according to any one of items 20 to 36, wherein the compound of formula 4 or the compound of formula 11 is reacted in step (ii) with 1,3-butanediol, 1,3-butanedithiol, 1-mercaptobutane-3-ol or 3-mercaptobutane-1-ol, resulting in X being

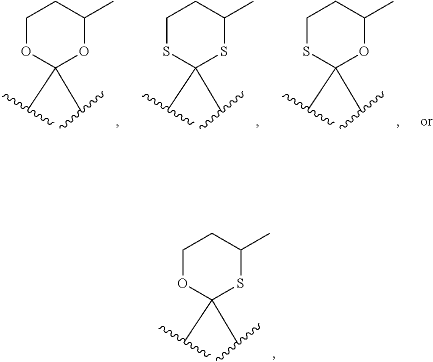

preferably wherein the compound of formula 4 or the compound of formula 11 is reacted in step (ii) with 1,3-butanediol resulting in X being

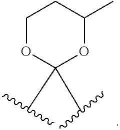 [SMILES: *C1(*)OCCC(C)O1]

38. The process according to any one of items 20 and 23 to 37, wherein the compound is selected from the group consisting of

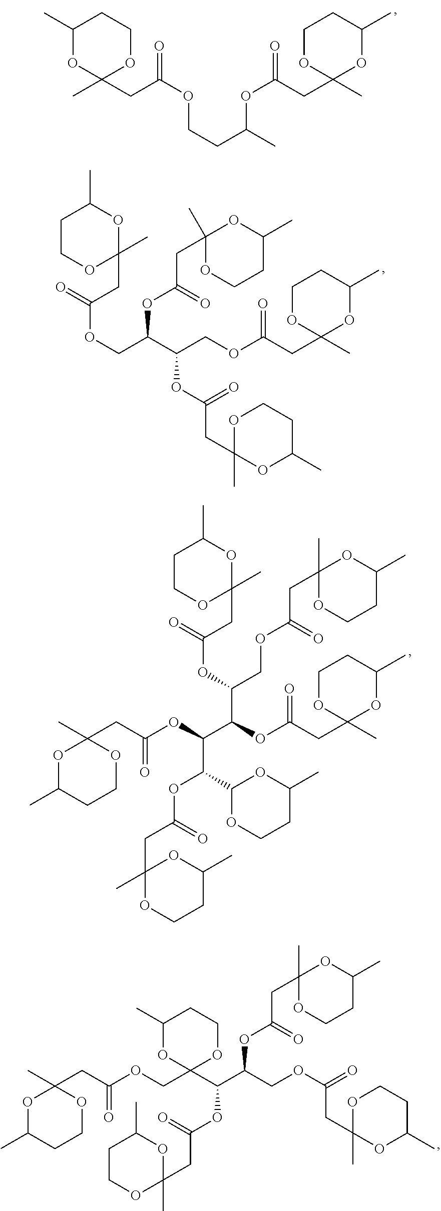

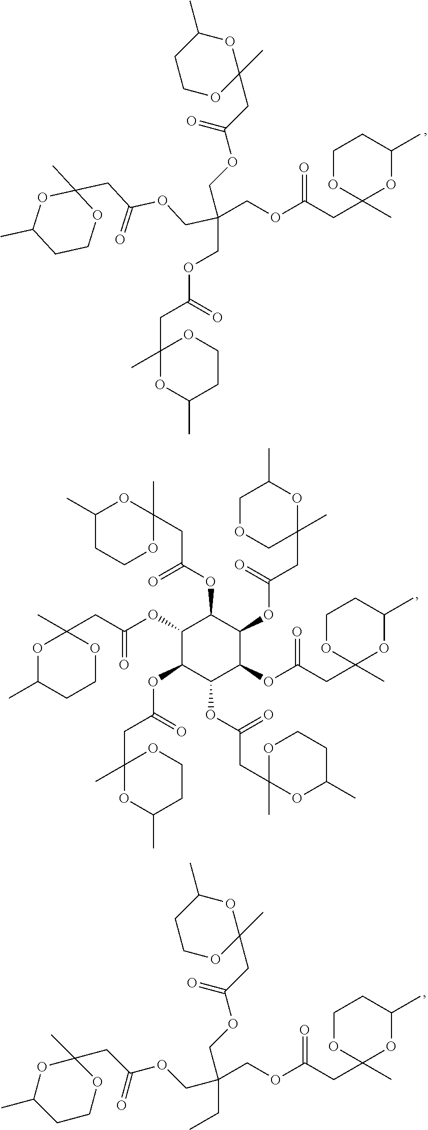

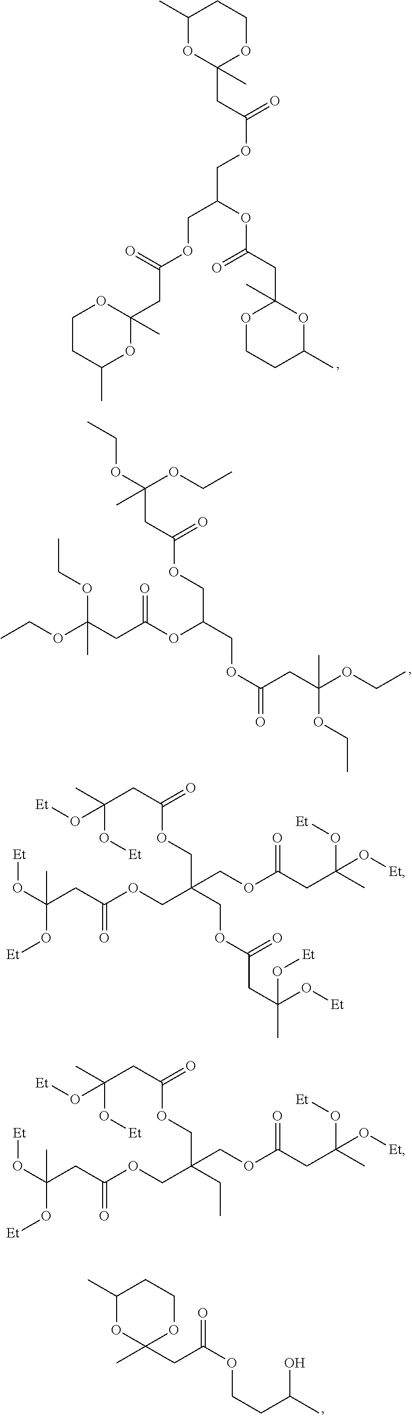

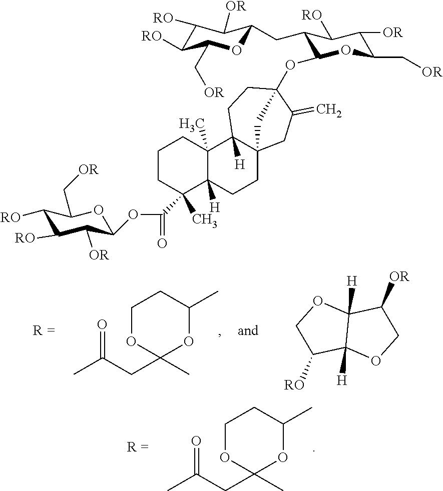

39. The process according any one of items 21 to 37, wherein the compound is selected from the group consisting of

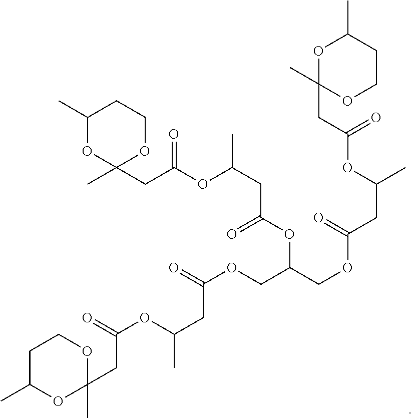 [SMILES: CC(CC(=O)OCC(COC(=O)CC(C)OC(=O)CC1(C)OCCC(C)O1)OC(=O)CC(C)OC(=O)CC1(C)OCCC(C)O1)OC(=O)CC1(C)OCCC(C)O1]

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention. All embodiments and preferred embodiments described herein in connection with one particular aspect of the invention (e.g. the inventive preservative composition) shall likewise apply to all other aspects of the present inventions such as end-use formulations, uses or methods according to the present invention.

The present invention will be further illustrated by the following examples.

### EXAMPLES

### Example 1

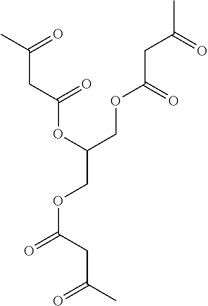 [SMILES: CC(=O)CC(=O)OCC(COC(=O)CC(C)=O)OC(=O)CC(C)=O]

Glycerol (650.0 g, 7.0 mol, 1 eq.) was introduced into a stirred tank reactor. DABCO (1.0 g, 9 mmol, 0.0013 eq.) was added and the mixture was stirred to obtain a homogenous mixture. Subsequently, diketene (1762.4 g, 21.0 mol, 3 eq. per hydroxyl group) was slowly dosed to the reaction mixture while cooling the reactor jacket to maintain an internal temperature of 40-70° C. The dosing rate was adjusted in order to maintain an internal temperature of 40-70° C. After complete addition, the mixture was maintained at an internal temperature of 40-70° C. for an additional 30 min. Finally, the reaction mixture was cooled to room temperature and analyzed. The final product propane-1,2,3-triyl tris(3-oxobutanoate) was obtained in quantitative yield. ¹H NMR (400 MHz, DMSO-d₆) δ ppm 2.18 (s, 9H) 3.60 (br d, J=6.90 Hz, 6H) 4.17-4.41 (m, 4H) 5.17-5.35 (m, 1H).

### Example 2

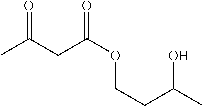

1,3-Butandiol (200.0 g, 2.2 mol, 1 eq.) was introduced into a stirred tank reactor. DABCO (0.3 g, 3 mmol, 0.0013 eq.) was added and the mixture was stirred to obtain a homogenous mixture. Subsequently, diketene (181.0 g, 2.15 mol, 0.97 eq. per hydroxyl group) was slowly dosed to the reaction mixture while cooling the reactor jacket to maintain an internal temperature of 40-70° C. The dosing rate was adjusted in order to maintain an internal temperature of 40-70° C. After complete addition, the mixture was maintained at an internal temperature of 40-70° C. for an additional 30 min. Finally, the reaction mixture was cooled to room temperature and analyzed. The final product 3-hydroxybutyl 3-oxobutanoate was obtained in quantitative yield. ¹H NMR (400 MHz, DMSO-d₆) δ ppm 0.98-1.13 (m, 3H) 1.06 (s, 1H) 1.62 (s, 2H) 2.18 (s, 3H) 3.56-3.65 (m, 2H) 3.65-3.79 (m, 1H) 3.95-4.21 (m, 2H) 4.42-4.65 (m, 1H).

### Example 3

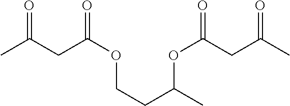 [SMILES: CC(=O)CC(=O)OCCC(C)OC(=O)CC(C)=O]

1,3-Butanediol (200.0 g, 2.2 mol, 1 eq.) was introduced into a stirred tank reactor. DABCO (0.3 g, 3 mmol, 0.0013 eq.) was added and the mixture was stirred to obtain a homogenous mixture. Subsequently, diketene (367.6 g, 4.4 mol, 2 eq. per hydroxyl group) was slowly dosed to the reaction mixture while cooling the reactor jacket to maintain an internal temperature of 40-70° C. The dosing rate was adjusted in order to maintain an internal temperature of 40-70° C. After complete addition, the mixture was maintained at an internal temperature of 40-70° C. for an additional 30 min. Finally, the reaction mixture was cooled to room temperature and analyzed. The final product butane-1,3-diyl bis(3-oxobutanoate) was obtained in quantitative yield. ¹H NMR (400 MHz, DMSO-d₆) δ ppm 1.21 (d, J=6.27 Hz, 3H) 1.79-1.92 (m, 2H) 2.18 (d, J=2.01 Hz, 6H) 3.59 (d, J=4.89 Hz, 4H) 4.12 (s, 2H) 4.86-4.99 (m, 1H).

### Example 4

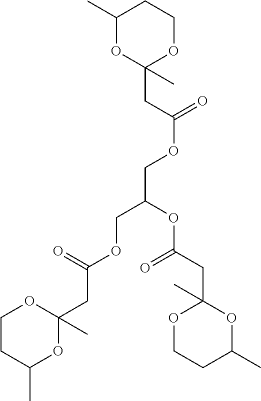 [SMILES: CC1CCOC(C)(CC(=O)OCC(COC(=O)CC2(C)OCCC(C)O2)OC(=O)CC2(C)OCCC(C)O2)O1]

Propane-1,2,3-triyl tris(3-oxobutanoate) (20.0 g, 58 mmol, 1 eq.) was introduced into a stirred tank reactor and dissolved in toluene (110 ml, 5.5 rel. vol.). P-toluenesulfonic acid (1.1 g, 6 mmol, 0.1 eq.) and 1,3-butandiol (63.1 g, 697 mmol, 12 eq) was added and the mixture was heated to reflux for 5 h. Water was removed with a dean-stark trap. After water formation ceased, the mixture was cooled to room temperature and the lower layer was discarded. The upper layer was extracted with NaHCO₃ sat. (75 ml) and water (30 ml) and concentrated to dryness under reduced pressure to obtain the final product propane-1,2,3-triyl tris(2-(2,4-dimethyl-1,3-dioxan-2-yl)acetate) in 87.5% yield. ¹H NMR (400 MHz, DMSO-d₆) δ ppm 0.97-1.25 (m, 18H) 1.41-1.49 (m, 3H) 1.56-1.66 (m, 3H) 2.47-2.56 (m, 3H) 2.71-3.05 (m, 3H) 3.63-3.73 (m, 3H), 3.84-4.17 (m, 10H) 4.79-5.00 (m, 1H).

### Example 5

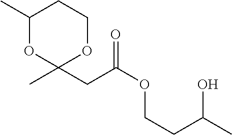 [SMILES: CC(O)CCOC(=O)CC1(C)OCCC(C)O1]

3-hydroxybutyl 3-oxobutanoate (50.0 g, 178 mmol, 1 eq.) was introduced into a stirred tank reactor and dissolved in toluene (75 ml, 1.5 rel. vol.). P-toluenesulfonic acid (0.17 g, 0.9 mmol, 0.005 eq.) and 1,3-butandiol (17.7 g, 196 mmol, 1.1 eq) was added and the mixture was heated to reflux for 5 h. Water was removed with a dean-stark trap. After water formation ceased, the mixture was cooled to room temperature and extracted with NaHCO₃ 5%-w/w (100 ml, 2 rel. vol.) and water (100 ml, 2 rel. vol.). The organic layer was dried over MgSO₄ and concentrated to dryness under reduced pressure to obtain the final product 3-hydroxybutyl 2-(2,4-dimethyl-1,3-dioxan-2-yl)acetate in 58.7% yield. ¹H NMR (400 MHz, DMSO-d₆) δ ppm 1.05 (d, J=5.90 Hz, 3H) 1.14-1.21 (m, 3H) 1.35 (s, 3H) 1.38-1.55 (m, 3H) 1.74-1.92 (m, 1H) 2.53 (s, 1H) 2.74-3.05 (m, 1H) 3.63-3.80 (m, 1H) 3.80-4.26 (m, 5H) 4.81-5.02 (m, 1H)

### Example 6

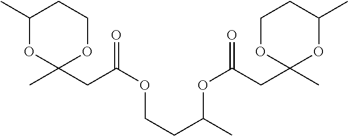

Butane-1,3-diyl bis(3-oxobutanoate) (50.0 g, 194 mmol, 1 eq.) was introduced into a stirred tank reactor and dissolved in toluene (125 ml, 2.5 rel. vol.). P-toluenesulfonic acid (0.19 g, 1 mmol, 0.005 eq.) and 1,3-butandiol (38.6 g, 426 mmol, 2.2 eq) was added and the mixture was heated to reflux for 5 h. Water was removed with a dean-stark trap. After water formation ceased, the mixture was cooled to room temperature and extracted with Na₂CO₃ 10%-w/w (100 ml, 2 rel. vol.) and water (100 ml, 2 rel. vol.). The organic layer was dried over MgSO₄ and concentrated to dryness under reduced pressure to obtain the final product butane-1,3-diyl bis(2-(2,4-dimethyl-1,3-dioxan-2-yl)acetate) in 61.4% yield. ¹H NMR (400 MHz, CHLOROFORM-d) δ ppm 1.04 (d, J=5.65 Hz, 6H) 1.12-1.26 (m, 3H) 1.26-1.72 (m, 9H) 1.74-1.92 (m, 2H) 2.50 (s, 2H) 2.71-3.04 (m, 2H) 3.63-3.77 (m, 2H) 3.81-4.19 (m, 5H) 4.77-5.00 (m, 1H).

### Example 7

Acetal cleavage in simulated gastric fluid (SGF): propane-1,2,3-triyl tris(2-(2,4-dimethyl-1,3-dioxan-2-yl)acetate) (1 g, 2 mmol, 1 eq) was mixed with simulated gastric fluid (SGF) (5 g, 5 rel. eq.) at 35-37° C. After 30 min, 1 h and 2 h the mixture was sampled and extracted with EtOAc. The extract was analyzed by thin layer chromatography and it was found that after 30 min a majority of the product had hydrolyzed to 1,3-butanediol and propane-1,2,3-triyl tris(3-oxobutanoate). After 1 h hydrolyzation was complete.

1.-16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A process for the preparation of a compound of formula 1 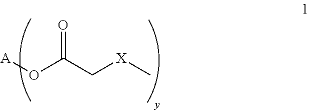
wherein A is derived from an organic polyol with at least 2 hydroxyl groups, y is from 2 to the number of hydroxyl groups of the initial organic polyol A, and X is 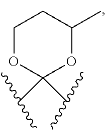 [SMILES: *C1(*)OCCC(C)O1]
the process comprising:
reacting an organic polyol of formula 2 with diketene 3 resulting in the formation of a compound according to formula 4; 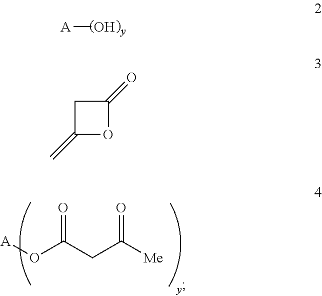
and
reacting the compound of formula 4 with 1,3-butandiol resulting in the formation of a compound according to formula 5

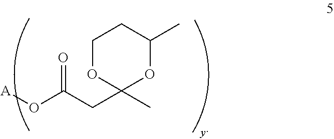

30. The process according to claim 29, wherein the organic polyol is selected from a linear or branched C₂₋₁₂ alkyl substituted with at least 2 hydroxyl groups or a C₃₋₈ cycloalkyl substituted with at least 2 hydroxyl groups.

31. The process according to claim 30, wherein one or both of:
the linear or branched C₂₋₁₂ alkyl substituted with at least 2 hydroxyl groups is selected from the group consisting of ethylene glycol, propanediol, glycerol, propanetriol, trimethylolpropane, 1,3-butanediol, butanediol, butanetriol, 2-methyl-propanetriol, pentanediol, pentanetriol, 3-methyl-pentanetriol hexanediol, hexanetriol, pentaerythritol, butanetetrol, pentanetetrol, hexanetetrol, hexanepentol, and combinations thereof; and
the C₃₋₈ cycloalkyl substituted with at least 2 hydroxyl groups is selected from the group consisting of cyclobutanediol, cyclopentanediol, cyclohexanediol, cyclopentanetriol, cyclohexanetriol, cyclopentanetetrol, cyclohexanetetrol, cyclopentanpentol, cyclohexanpentol, cyclohexanhexol, dihydroxytetrahydrofuran, trihydroxytetrahydrofuran, tetrahydroxytetrahydrofuran, dihydroxytetrahydropyrane, trihydroxytetrahydropyrane, tetrahydroxytetrahydropyrane, isosorbide, and combinations thereof.

32. The process according to claim 30, wherein the linear or branched C₂₋₁₂ alkyl substituted with at least 2 hydroxyl groups is selected from the group consisting of glycerol and 1,3-butanediol.

33. The process according to claim 30, wherein the organic polyol is selected from the group consisting of monosaccharides, disaccharides, oligosaccharides, sugar alcohols, and sugar acids.

34. The process according to claim 33, wherein:
the monosaccharide is selected from trioses, tetroses, pentoses, hexoses, and heptoses;
the monosaccharide is selected from aldotrioses, ketotrioses, aldotetroses, ketotetroses, aldopentoses, ketopentoses, aldohexosen, ketohexoses, aldoheptoses and ketoheptoses; or
the monosaccharide is selected from the group consisting of glyceraldehyde, dihydroxyacetone, erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

35. The process according to claim 33, wherein the disaccharide is selected from the group consisting of sucrose, sucralose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, mannobiose, melibiose, melibiulose, rutinose, rutinulose, xylobiose, and combinations thereof.

36. The process according to claim 33, wherein the oligosaccaride is selected from the group consisting of stevioside, steviol glycoside, raubaudioside A, raubaudioside B, raubaudioside C, raubaudioside D, raffinose, and combinations thereof.

37. The process according to claim 33, wherein one or both of:
the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and combinations thereof; and
wherein the sugar acid is selected from the group consisting of glyceric acid, tartaric acid, xylonic acid, gluconic acid, ascorbic acid, neuraminic acid, ketodeoxyoctonic acid, glucuronic acid, galacturonic acid, iduronic acid, mucic acid, saccharic acid, and combinations thereof.

38. The process according to claim 30, wherein the organic polyol is selected from the group consisting of 1,3-butanediol, glycerol, sorbitol, xylitol, mannitol, erythritol, maltitol, glucose, glucitol, ribulose, pentaerythritol, trimethylolpropane, stevioside, and isosorbide, preferably wherein the organic polyol is glycerol or 1,3-butanediol, even more preferably wherein the organic polyol is 1,3-butanediol.

39. The process according to claim 30, wherein y is equal to the number of hydroxyl groups of the initial polyol A.

40. The process according to claim 30, wherein the compound is selected from the group consisting of

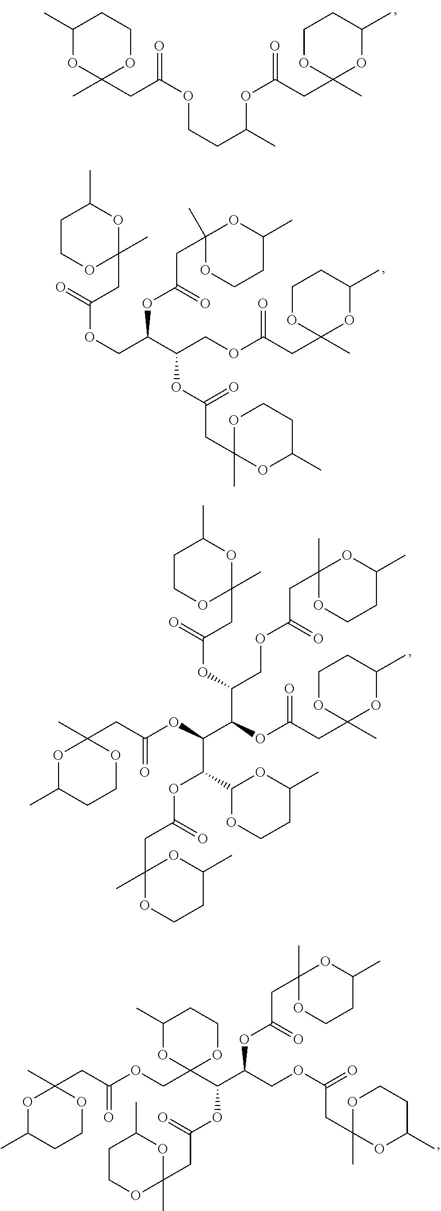

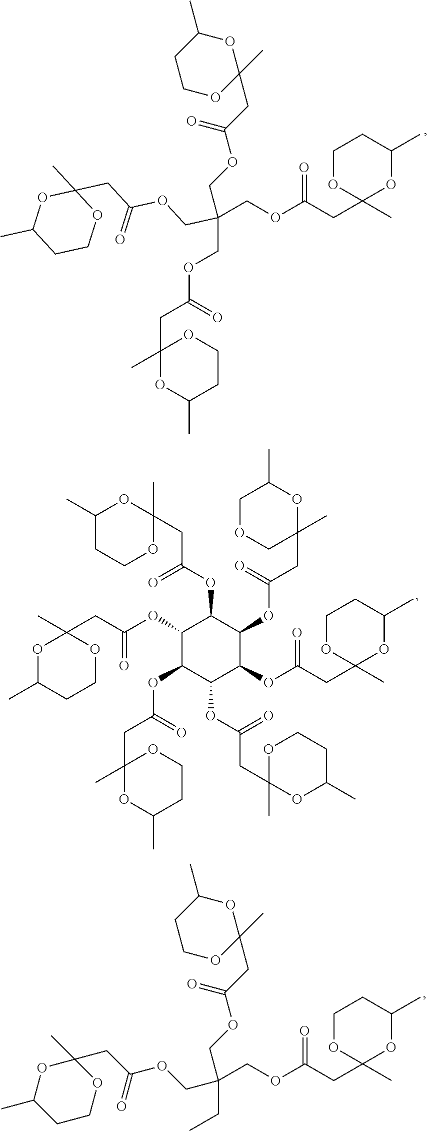

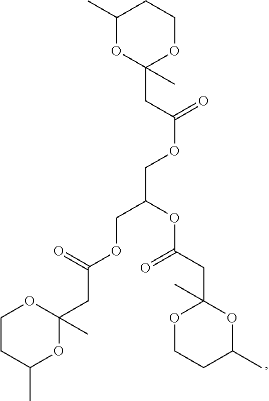 [SMILES: CC1CCOC(C)(CC(=O)OCC(COC(=O)CC2(C)OCCC(C)O2)OC(=O)CC2(C)OCCC(C)O2)O1]

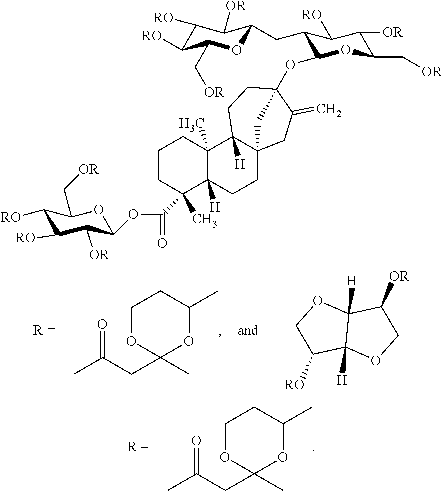

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A process for the preparation of a compound of formula 9 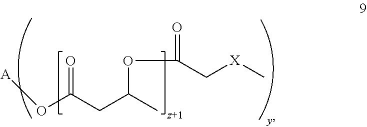
wherein z is 0 or more,
wherein A is derived from an organic polyol with at least 2 hydroxyl groups,
wherein y is from 2 to the number of hydroxyl groups of the initial organic polyol A, and
wherein X is 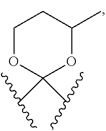 [SMILES: *C1(*)OCCC(C)O1]
the process comprising:
reacting a compound of formula 10 with diketene 3 resulting in the formation of a compound according to formula 11; 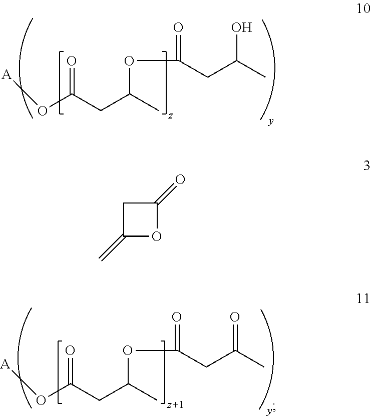
and
reacting the compound of formula 11 with 1,3-butandiol resulting in the formation of a compound according to formula 12

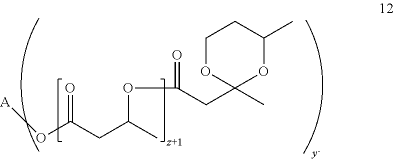

55. The process according to claim 54, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.

56. The process according to claim 54, wherein the organic polyol is selected from a linear or branched C₂₋₁₂ alkyl substituted with at least 2 hydroxyl groups or a C₃₋₈ cycloalkyl substituted with at least 2 hydroxyl groups.

57. The process according to claim 56, wherein one or both of:
the linear or branched C₂₋₁₂ alkyl substituted with at least 2 hydroxyl groups is selected from the group consisting of ethylene glycol, propanediol, glycerol, propanetriol, trimethylolpropane, 1,3-butanediol, butanediol, butanetriol, 2-methyl-propanetriol, pentanediol, pentanetriol, 3-methyl-pentanetriol hexanediol, hexanetriol, pentaerythritol, butanetetrol, pentanetetrol, hexanetetrol, hexanepentol, and combinations thereof; and
the C₃₋₈ cycloalkyl substituted with at least 2 hydroxyl groups is selected from the group consisting of cyclobutanediol, cyclopentanediol, cyclohexanediol, cyclopentanetriol, cyclohexanetriol, cyclopentanetetrol, cyclohexanetetrol, cyclopentanpentol, cyclohexanpentol, cyclohexanhexol, dihydroxytetrahydrofuran, trihydroxytetrahydrofuran, tetrahydroxytetrahydrofuran, dihydroxytetrahydropyrane, trihydroxytetrahydropyrane, tetrahydroxytetrahydropyrane, isosorbide, and combinations thereof.

58. The process according to claim 56, wherein the linear or branched C₂₋₁₂ alkyl substituted with at least 2 hydroxyl groups is selected from the group consisting of glycerol and 1,3-butanediol.

59. The process according to claim 54, wherein the organic polyol is selected from the group consisting of monosaccharides, disaccharides, oligosaccharides, sugar alcohols, and sugar acids.

60. The process according to claim 59, wherein:
the monosaccharide is selected from trioses, tetroses, pentoses, hexoses, and heptoses;
the monosaccharide is selected from aldotrioses, ketotrioses, aldotetroses, ketotetroses, aldopentoses, ketopentoses, aldohexosen, ketohexoses, aldoheptoses and ketoheptoses; or
the monosaccharide is selected from the group consisting of glyceraldehyde, dihydroxyacetone, erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

61. The process according to claim 59, wherein the disaccharide is selected from the group consisting of sucrose, sucralose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, mannobiose, melibiose, melibiulose, rutinose, rutinulose, xylobiose, and combinations thereof.

62. The process according to claim 59, wherein the oligosaccaride is selected from the group consisting of stevioside, steviol glycoside, raubaudioside A, raubaudioside B, raubaudioside C, raubaudioside D, raffinose, and combinations thereof.

63. The process according to claim 59, wherein one or both of:
the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and combinations thereof; and
wherein the sugar acid is selected from the group consisting of glyceric acid, tartaric acid, xylonic acid, gluconic acid, ascorbic acid, neuraminic acid, ketodeoxyoctonic acid, glucuronic acid, galacturonic acid, iduronic acid, mucic acid, saccharic acid, and combinations thereof.

64. The process according to claim 54, wherein the organic polyol is selected from the group consisting of 1,3-butanediol, glycerol, sorbitol, xylitol, mannitol, erythritol, maltitol, glucose, glucitol, ribulose, pentaerythritol, trimethylolpropane, stevioside, and isosorbide, preferably wherein the organic polyol is glycerol or 1,3-butanediol, even more preferably wherein the organic polyol is 1,3-butanediol.

65. The process according to claim 54, wherein y is equal to the number of hydroxyl groups of the initial polyol A.

66. The process according to claim 54, wherein the compound is 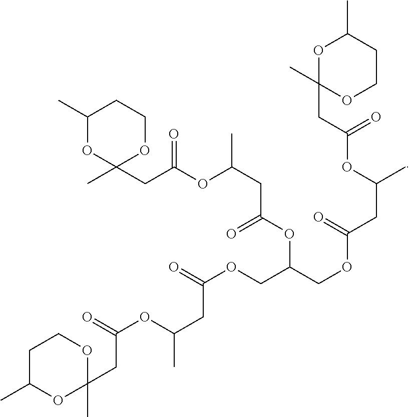 [SMILES: CC(CC(=O)OCC(COC(=O)CC(C)OC(=O)CC1(C)OCCC(C)O1)OC(=O)CC(C)OC(=O)CC1(C)OCCC(C)O1)OC(=O)CC1(C)OCCC(C)O1]
